(19) United States
(12) Patent Application Publication
(10) Pub. No.: US 2014/0005797 A1
(43) Pub. Date: Jan. 2, 2014

(54) THREE-DIMENSIONAL POROUS SCAFFOLD AND MANUFACTURING METHOD THEREOF

(71) Applicants: Young Hwan Park, Seongnam-si (KR); Jung Nam Im, Gunpo-si (KR); Tae Hee Kim, Suwon-si (KR); Jae Hoon Ko, Ansan-si (KR); Sung Jin Kim, Gumi-si (KR)

(72) Inventors: Young Hwan Park, Seongnam-si (KR); Jung Nam Im, Gunpo-si (KR); Tae Hee Kim, Suwon-si (KR); Jae Hoon Ko, Ansan-si (KR); Sung Jin Kim, Gumi-si (KR)

(21) Appl. No.: 13/984,431

(22) PCT Filed: Mar. 15, 2012
(86) PCT No.: PCT/KR12/01873

§ 371 Date: Sep. 13, 2013

(30) Foreign Application Priority Data
May 26, 2011 (KR) 10-2011-0049996

(51) Int. Cl. A61L 27/56; A61L 27/58

(52) U.S. Cl. CPC A61L 27/56; A61L 27/58
USPC 623/23.72; 264/103

(57) ABSTRACT

A three-dimensional porous scaffold and a preparation method thereof. The three-dimensional porous scaffold comprises a biodegradable multifilament draw-textured yarn on the inside of a tubular knitted fabric made of a biodegradable polymer. The three-dimensional porous scaffold has a porosity formed by the network mesh structure of the tubular knitted fabric and the 10-150 μm pores formed in the biodegradable multifilament draw-textured yarn, while it has a bulkiness of 150-1000% due to the biodegradable multifilament drawn textured yarn inserted in the tubular knitted fabric. Thus, the scaffold has a high degree of interconnection between pores, so that cell culture, cell delivery or drug delivery on the stable three-dimensional scaffold structure is performed in an optimized manner.

### CROSS-REFERENCE TO RELATED APPLICATIONS

This application is a National Stage application of International Application No. PCT/KR2012/001873, filed on Mar. 15, 2012, which claims priority of Korean application Serial Number 10-2011-0049996 filed on May 26, 2011, which are incorporated herein by reference in their entireties.

### BACKGROUND OF THE INVENTION

1. Field of the Invention

The present invention relates to a three-dimensional porous scaffold and a manufacturing method thereof. More particularly, the present invention relates to a three-dimensional porous scaffold, which comprises a biodegradable multifilament draw-textured yarn on the inside of a tubular knitted fabric made of a biodegradable polymer and performs cell culture, cell delivery or drug delivery in an optimized manner, and a manufacturing method thereof.

2. Description of the Prior Art

As used herein, the term “scaffold” refers to a material that can regenerate tissue in a human body damage area caused by an accident or disease or provide a support.

Generally, scaffolds are prepared by a fiber knitting method, a fiber adhesion method, a solvent casting method, a particulate leaching method, a melting molding method, a membrane lamination method, an extrusion molding method, a freeze drying method, an emulsion freeze drying method, a phase separation method, a foam molding method utilizing gas, an electrospinning method or the like.

Among the above preparation methods, the fiber knitting method, the fiber adhesion method, the membrane lamination method and the electrospinning method have a shortcoming in that transplanted cells grow in a two-dimensional manner, because a web-like scaffold is prepared.

On the other hand, the solvent casting method, the particulate leaching method, the extrusion molding method, the freeze drying method, the phase separation method and the foam molding method utilizing gas can prepare three-dimensional scaffolds, but the scaffolds have a poor degree of interconnections between the pores of the scaffolds, and thus the metabolism of transplanted cells in a culture process does not easily occur so that the transplanted cells are very difficult to grow and differentiate.

Accordingly, the present inventors have made extensive efforts regarding the above-described problems occurring in the prior art, and as a result, have developed a three-dimensional porous scaffold suitable for cell culture, cell delivery or drug delivery by inserting a bulky biodegradable multifilament draw-textured yarn into a tubular knitted fabric made of a biodegradable polymer so that the scaffold has a high degree of interconnection between the pores of the scaffold, thereby completing the present invention.

### SUMMARY OF THE INVENTION

Accordingly, it is an object of the present invention to provide a three-dimensional porous scaffold comprising a biodegradable fiber having a bulky structure.

Another object of the present invention is to provide a method for preparing a three-dimensional porous scaffold, which comprises inserting a biodegradable multifilament draw-textured yarn into a tubular knitted fabric made of a biodegradable polymer and drawing the inserted biodegradable multifilament draw-textured yarn.

To achieve the above objects, the present invention provides a three-dimensional porous scaffold comprising a biodegradable multifilament draw-textured yarn, which has a bulkiness of 150-1000%, on the inside of a tubular knitted fabric made of a biodegradable polymer.

In the three-dimensional porous scaffold of the present invention, the tubular knitted fiber made of the biodegradable polymer is preferably a 1-50 denier monofilament fiber, a 100-500 denier multifilament fiber or a spun fiber. Preferably, the biodegradable polymer that is used in the present invention may be one or more selected from the group consisting of polylactic acid, polyglycolic acid, poly-ε-caprolactone, polylactic acid-co-glycolic acid, poly-3-hydroxybutyrate (PHB), polyhydroxyvalerate (PHV) and polyhydroxybutyrate-co-valerate (PHBV).

In the three-dimensional porous scaffold of the present invention, the tubular knitted fabric has a cross-sectional diameter of 5-20 mm and has a network mesh structure.

Also, in the three-dimensional porous scaffold of the present invention, the biodegradable multifilament draw-textured yarn is made of a biodegradable synthetic polymer of a homopolymer or copolymer selected from the group consisting of polylactic acid, polyglycolic acid, polycaprolactone, polylactic acid-co-glycolic acid, poly-3-hydroxybutyrate(PHB), polyhydroxyvalerate(PHV), polyhydroxybutyrate-co-valerate(PHBV), dioxanone, trimethylene carbonate and ethylene oxide, or a biodegradable natural polymer selected from among collagen, cellulose oxide, chitosan, chitin, gelatin and silk fibroin.

More preferably, the biodegradable multifilament draw-textured yarn is made of a polylactic acid-co-glycolic acid obtained by copolymerizing lactide and with glycolide at a weight ratio of 10:90 to 30:70.

The biodegradable multifilament draw-textured yarn includes pores having a size of 10-150 μm.

The present invention also provides a method for preparing a three-dimensional porous scaffold, the method comprising: introducing a plied multifilament yarn made of a biodegradable polymer into a fine knitting machine to prepare a tubular knitted fabric; spinning a biodegradable polymer into a monofilament or multifilament yarn by a melt-spinning or wet-spinning process and ply-twisting the spun yarn to prepare a biodegradable multifilament draw-textured yarn; inserting the biodegradable multifilament draw-textured yarn into the tubular knitted fabric; and drawing the inserted biodegradable multifilament draw-textured yarn to impart bulkiness.

In the preparation method of the present invention, the multifilament yarn has a single-yarn diameter of 5-30 μm, and the plied multifilament yarn has a diameter of 80-8000 μm.

In the preparation method of the present invention, pores having a size of 10-150 μm and bulkiness are imparted to the biodegradable multifilament draw-textured yarn by the drawing. As used herein, the term “bulkiness” means that the volume of the biodegradable multifilament draw-textured yarn is partially increased by 150-1000% over non-bulky biodegradable multifilament draw-textured yarn.

According to the present invention, a three-dimensional porous scaffold comprising a biodegradable fiber having a bulky structure can be provided.

The three-dimensional porous scaffold of the present invention is prepared such that a biodegradable multifilament draw-textured yarn having bulkiness is inserted in a tubular knitted fabric made of a biodegradable polymer. The biodegradable multifilament draw-textured yarn inserted in the tubular knitted fabric imparts a bulkiness of 150-1000%, and thus the scaffold has a high degree of interconnection between pores so that it can be advantageously used for cell culture, cell delivery or drug delivery on the stable three-dimensional scaffold structure.

### BRIEF DESCRIPTION OF THE DRAWINGS

FIG. 1 shows each step of the inventive method for preparing a three-dimensional porous scaffold.

FIG. 2 is a photograph of the side of a tubular knitted fabric prepared in Example 1 of the present invention, taken along the lengthwise direction of the knit.

FIG. 3 is a top view of the tubular knitted fabric shown in FIG. 2.

FIG. 4 shows the results of measurement of the pore size distribution of the tubular knitted fabric shown in FIG. 3.

### DETAILED DESCRIPTION OF THE PREFERRED EMBODIMENT

Hereinafter, the present invention will be described in detail.

The present invention provides a three-dimensional porous scaffold, wherein a biodegradable multifilament draw-textured yarn having a bulkiness of 150-1000% is inserted in a tubular knitted fabric made of a biodegradable polymer so that the draw-textured yarn can be used for cell culture, cell delivery or drug delivery.

The three-dimensional scaffold of the present invention has a porosity formed by the surface of the network mesh structure of the tubular knitted fabric made of the biodegradable polymer and the 10-150 μm pores formed in the biodegradable multifilament textured yarn.

Hereinafter, each element of the three-dimensional porous scaffold of the present invention will be described.

1) Tubular Knitted Fabric Made of Biodegradable Polymer

The inventive tubular knitted fabric made of the biodegradable polymer is made of a synthetic or natural biodegradable polymer, which can be spun into a 1-50 denier monofilament or a 100-500 denier multifilament fiber by a melt-spinning or wet-spinning process, or a synthetic or natural staple spun fiber. Preferably, it is a fiber knitted to a thickness of 100-500 denier using a fine knitting machine.

Herein, the biodegradable polymer material should be a material harmless to the human body even when being inserted into or attached to the body. Preferably, the biodegradable polymer is at least one selected from the group consisting of polylactic acid (PLA), polyglycolic acid (PGA), poly-ε-caprolactone (PCL), polylactic acid-co-glycolic acid (PLGA), poly-3-hydroxybutyrate (PHB), polyhydroxyvalerate (PHV) and polyhydroxybutyrate-co-valerate (PHBV).

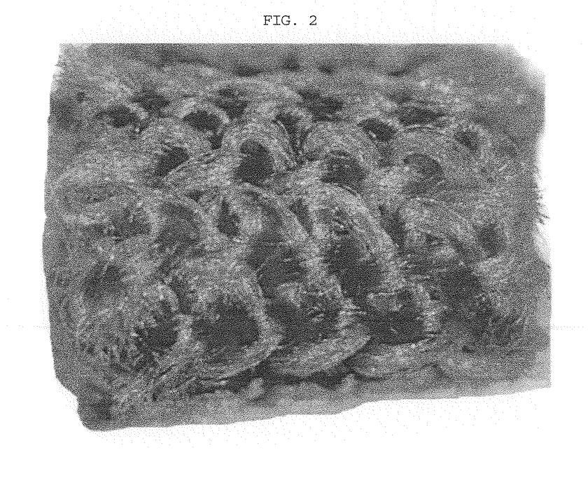

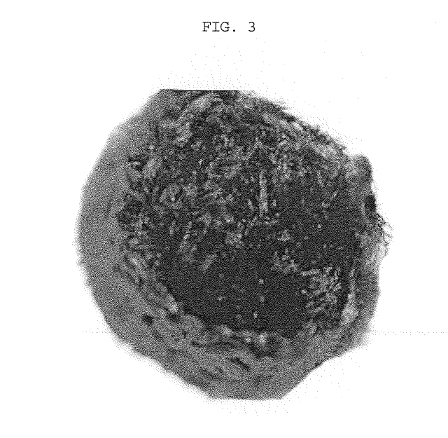

FIG. 2 is a photograph of the side of a tubular knitted fabric of the present invention, taken along the lengthwise direction of the knit, and FIG. 3 is a top view of the tubular knitted fabric. The tubular knitted fabric prepared in Example 1 has a diameter of 8 mm and a length of 100 mm.

The tubular knitted fabric of the present invention has a network mesh structure. A tubular knitted fabric consisting of a fine yarn will have large pores between nets, and a tubular knitted fabric consisting of a coarse yarn will have small pores between nets. Thus, the pore size can be controlled according to the structure of the tubular knitted fabric.

Preferably, the tubular knitted fabric has an approximately circular cross-section and a diameter of 5-20 mm, and more preferably 5-12 mm.

If the diameter of the circular knitted fabric is less than 5 mm, the bulkiness of the biodegradable multifilament draw-textured yarn on the inside of the tubular knitted fabric will be insufficient, and thus the efficiency of cell culture, cell delivery or drug delivery will be reduced. On the other hand, if the diameter of the circular knitted fabric is more than 20 mm, the internal space of the tubular knitted fabric and the distance between meshes in the tubular knitted fabric will be excessively increased, and thus the degree of interconnection between the pores will be reduced so that the ability to retain cells or drugs will be reduced.

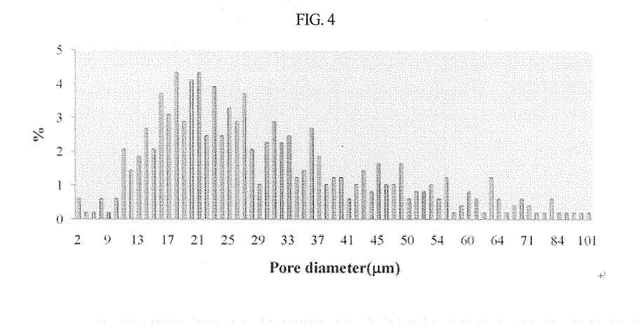

FIG. 4 shows the results of measurement of the pore size distribution of the tubular knitted fabric of the present invention.

2) Biodegradable Multifilament Draw-Textured Yarn Having Bulkiness

The three-dimensional porous scaffold of the present invention performs cell culture, cell delivery or drug delivery in the bulky structure of the biodegradable multifilament draw-textured yarn inserted in the circular knitted fabric.

The biodegradable multifilament draw-textured yarn of the present invention is a network structure having pores in the bulky structure, and the bulkiness or the pore size can be controlled depending on drawing conditions during the preparation of the draw-textured yarn.

In other words, according to the present invention, a bulkiness of 150-1000% is further imparted to a biodegradable multifilament draw textured yarn having inherent bulkiness and a soft feeling, so that the bulky structure is suitable for cell culture, cell delivery or drug delivery when it is used in medical applications.

Further, the present invention provides a three-dimensional porous scaffold wherein the biodegradable multifilament draw-textured yarn having bulkiness is inserted in the tubular knitted fabric.

As used herein, the term “bulky structure” refers to a structure in which a plurality of pores having a size of 1 μm or more are present between fibers, and the term “bulkiness” means that a growing volume of 150-1000% is imparted to the biodegradable multifilament draw-textured yarn made of the biodegradable polymer after the preparation of the yarn by drawing or stretching.

The bulkiness of the biodegradable multifilament draw-textured yarn of the present invention can be freely controlled depending on the intended use, such as cell culture, cell delivery or drug delivery. However, if the bulkiness is less than 150%, the pore size of the yarn will be reduced, and thus cells in the scaffold will be difficult to proliferate during cell culture and the content of cells or drugs that can be delivered in vivo will be reduced, indicating that the efficiency of the scaffold in medical applications is low. On the other hand, if the bulkiness is more than 1000%, the rate of occurrence of yarn breakage will be increased due to the low durability of the biodegradable polymer resin, and the pore size of the yarn will be excessively increased to reduce the ability to retain cells or drugs.

It is to be understood that the size of pores in the biodegradable multifilament draw-textured yarn of the present invention can be suitably controlled depending on the size of the selected cell or drug. Specifically, the bulky structure of the multifilament draw-textured yarn of the present invention has a pore size of 1-150 μm, and preferably 5-50 μm. If the pore size is less than 1 cells in the bulky structure will be difficult to proliferate during cell culture, and the content of cells or drugs that can be delivered in vivo will be reduced, indicating that the efficiency of the scaffold in medical applications is low. One the other hand, if the pore size is more than 150 μm, the ability to retain cells or drugs will be reduced.

The biodegradable multifilament draw-textured yarn having bulkiness according to the present invention is harmless to the human body even when being inserted into or patched to the body, and it must be able to be absorbed in vivo after it was used for cell culture, cell delivery or drug delivery. Thus, the biodegradable multifilament draw-textured yarn should be made of a synthetic or natural biodegradable polymer which can be spun into a monofilament or multifilament fiber by a melt-spinning or wet-spinning process. Specifically, a monofilament or multifilament yarn is plied to a thickness of 50-500 denier, and the plied yarn is passed through a twisting machine such as a roller-type or disc-type twisting machine and twisted in the S-direction or Z-direction so that partial bulkiness is imparted.

The draw-textured yarn is made of a biocompatible natural or synthetic polymer. Preferably, it is made of a biodegradable synthetic polymer of a homopolymer or copolymer selected from the group consisting of polylactic acid(PLA), polyglycolic acid(PGA), poly ε-caprolactone(PCL), polylactic acid-co-glycolic acid(PLGA), poly-3-hydroxybutyrate (PHB), polyhydroxyvalerate(PHV), polyhydroxybutyrate-co-valerate (PHBV), dioxanone, trimethylene carbonate and ethylene oxide, or a biodegradable natural polymer selected from among collagen, cellulose oxide, chitosan, chitin, gelatin and silk fibroin.

More preferably, the biodegradable multifilament draw-textured yarn is made of a polylactic acid-co-glycolic acid obtained by copolymerizing lactide with glycolide at a weight ratio of 10:90 to 30:70. The examples of the present invention illustrate the use of a polylactic acid-co-glycolic acid obtained by copolymerizing lactide with glycolide at a weight ratio of 10:90, but the scope of the present invention is not limited to the above weight ratio or material.

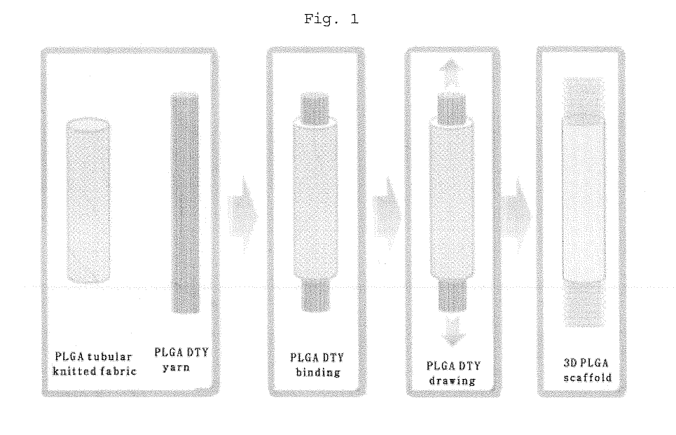

FIG. 1 shows each step of the inventive method for preparing a three-dimensional porous scaffold. As shown in FIG. 1, the present invention provides a method for preparing a three-dimensional porous scaffold, the method comprising: 1) introducing a plied multifilament yarn made of a biodegradable polymer into a fine knitting machine to prepare a tubular knitted fabric; 2) spinning a biodegradable polymer into a monofilament or multifilament yarn by a melt-spinning or wet-spinning process and ply-twisting the spun yarn to prepare a biodegradable multifilament draw-textured yarn; 3) inserting the biodegradable multifilament draw-textured yarn of step 2) into the tubular knitted fabric of step 1); and 4) drawing the inserted biodegradable multifilament draw-textured yarn to impart bulkiness.

In the inventive method for preparing the three-dimensional porous scaffold, the tubular knitted fabric prepared in step 1) is a fiber having an approximately circular cross section and has a length of 50-120 mm and a diameter of 5-20 mm, and preferably 5-12 mm.

The biodegradable polymer that is used in the method of the present invention is a material harmless to the human body even when being inserted into or to patched to the body, and specific examples thereof are as described above.

Step 2) of the method of the present invention is a step of preparing the biodegradable multifilament draw-textured yarn. In this step, the biodegradable natural polymer is spun through a spinneret to prepare having a single-yarn diameter of 5-30 μm. The biodegradable multifilament yarn prepared in step 2) is an ultrafine fiber having a diameter of 30 μm or less and satisfies a tenacity of 2.0-9.0 g/d and an elongation of 20-80%, and thus the occurrence of yarn breakage and the deterioration in quality during the subsequent drawing process can be minimized. Meanwhile, if the single-yarn diameter of the biodegradable multifilament draw-textured yarn is more than 30 μm, the rate of degradation of the draw-textured yarn after cell culture, cell delivery or drug delivery can be reduced, and the stiffness of the draw-textured yarn will increase to reduce the workability or operational convenience thereof.

When the scaffold of the present invention is applied as a scaffold in the human body, the diameter of the multifilament yarn after plying is preferably 80-8000 μm, and more preferably 1000-4000 μm. If the diameter of the plied yarn is less than 80 μm, it will be difficult for the resulting medical scaffold to have a 3-dimensional structure, and if the diameter is more than 8000 μm, the foreign body reaction of the polymer in vivo will increase.

In step 3) of the method of the present invention, the biodegradable multifilament draw-textured yarn prepared in step 2) is prepared to have a length corresponding to 1-3 times the length of the tubular knitted fabric and is inserted into the tubular knitted fabric.

Step 4) of the method of the present invention is a step of drawing the inserted biodegradable multifilament yarn to impart bulkiness. In this step, pores having a size of 10-150 μm and bulkiness are imparted to the biodegradable multifilament draw-textured yarn by the drawing process.

As used herein, the term “bulkiness” means that the volume of the biodegradable multifilament draw-textured yarn is increased by 150-1000% over non-bulky biodegradable multifilament draw-textured yarn.

In the preparation method of the present invention, the bulky structure is imparted by winding the biodegradable multifilament draw-textured yarn on a stretchable rack and then drawing the draw-textured yarn at a ratio of 5-20%. If the drawing ratio is less than 5%, it will be difficult to make the bulky structure, and if the drawing ratio is more than 20%, yarn breakage will be likely to occur.

Another method for imparting the bulky structure is performed by a drawing method in a continuous process.

Hereinafter, the present invention will be described in further detail with reference to examples. It is to be understood, however, that these examples are for illustrative purposes only and are not intended to limit the scope of the present invention.

### EXAMPLE 1

A plied yarn consisting of 4-multifilament yarns made of a polylactic acid-co-glycolic acid (PLGA) chip obtained by copolymerizing lactide with glycolide at a weight ratio of 10:90 was introduced into a fine knitting machine to prepare a tubular knitted fabric having a diameter of 8 mm and a length of 100 mm.

A polylactic acid-glycolic acid (PLGA) copolymer chip consisting of lactide and glycolide at a weight ratio of 10:90 was spun into a multifilament yarn of PLGA (10:90) consisting of 15 filaments each having a diameter of 50 denier by a melt-spinning process. Four fiber yarns were plied, and then introduced into a roller-type twisting machine to prepare a draw-textured yarn (DTY) having a Z-direction twist.

The DTY yarn of PLGA (200 denier and 64 filaments) was plied to obtain a 64-ply DTY yarn, and then inserted into the above-prepared tubular knitted fabric PLGA (10:90). Then, the DTY yarn of PLGA (200 denier and 64 filaments) was drawn at a ratio of 15% to prepare a three-dimensional porous scaffold which was then cut to a size of 10 mm so as to be easily used in a 24-well microplate culture dish. The prepared scaffold had a bulky structure and thus a high degree of interconnection between pores, and the scaffold having the bulky structure had a bulkiness of 500%.

### EXAMPLES 2 to 6

Three-dimensional porous scaffolds were prepared in the same manner as described in Example 1, except that the DTY yarn of PLGA (200 denier and 64 filaments) was plied to obtain each of 16-ply, 32-ply, 100-ply, 150-ply and 200-ply DTY yarns.

As described above, the present invention provides a three-dimensional porous scaffold comprising a bulky multifilament draw-textured yarn inserted in a tubular knitted fabric made of a biodegradable polymer.

The three-dimensional porous scaffold of the present invention has a porosity formed by the network mesh structure of the tubular knitted fabric and the 10-150 μm pores formed in the biodegradable multifilament draw-textured yarn, while it has a bulkiness of 150-1000% due to the biodegradable multifilament drawn textured yarn inserted in the circular knitted fabric. Thus, the scaffold has a high degree of interconnection between pores, so that cell culture, cell delivery or drug delivery on the stable three-dimensional scaffold structure is performed in an optimized manner.

Although the preferred embodiments of the present invention have been described for illustrative purposes, those skilled in the art will appreciate that various modifications, additions and substitutions are possible, without departing from the scope and spirit of the invention as disclosed in the accompanying claims.

We claim:

1. A three-dimensional porous scaffold comprising a biodegradable multifilament draw-textured yarn, which has a bulkiness of 150-1000%, on the inside of a network mesh structured tubular knitted fabric made of a biodegradable polymer.

2. The three-dimensional porous scaffold according to claim 1, wherein the tubular knitted fabric made of the biodegradable polymer is selected from the group consisting of a monofilament fiber a multifilament fiber and a spun fiber.

3. The three-dimensional porous scaffold according to claim 1, wherein the biodegradable polymer is one or more selected from the group consisting of polylactic acid, polyglycolic acid, poly-ε-caprolactone, polylactic acid-co-glycolic acid, poly-3-hydroxybutyrate, polyhydroxyvalerate and polyhydroxybutyrate-co-valerate.

4. The three-dimensional porous scaffold according to claim 1, wherein the tubular knitted fabric has a cross-sectional diameter of 5-20 mm.

5. (canceled)

6. The three-dimensional porous scaffold according to claim 1, wherein the biodegradable multifilament draw-textured yarn is made of a biodegradable synthetic polymer of a homopolymer or copolymer selected from the group consisting of polylactic acid, polyglycolic acid, polycaprolactone, polylactic acid-co-glycolic acid, poly-3-hydroxybutyrate, polyhydroxyvalerate, polyhydroxybutyrate-co-valerate, dioxanone, trimethylene carbonate and ethylene oxide, or a biodegradable natural polymer selected from the group consisting of collagen, cellulose oxide, chitosan, chitin, gelatin and silk fibroin.

7. The three-dimensional porous scaffold according to claim 1, wherein the biodegradable multifilament draw-textured yarn is made of a polylactic acid-co-glycolic acid obtained by copolymerizing lactide with glycolide at a weight ratio of 10:90 to 30:70.

8. The three-dimensional porous scaffold according to claim 1, wherein the biodegradable multifilament draw-textured yarn includes pores having a size of 10-150 μm.

9. A method for preparing a three-dimensional porous scaffold, the method comprising the steps of:
introducing a plied multifilament yarn made of a biodegradable polymer into a fine knitting machine to prepare a tubular knitted fabric having a network mesh structure;
spinning a biodegradable polymer into a monofilament or multifilament yarn by a melt-spinning or wet-spinning process to form a spun yarn, and then plying and twisting the spun yarn to prepare a biodegradable multifilament draw-textured yarn;
inserting the biodegradable multifilament draw-textured yarn into the tubular knitted fabric having a network mesh structure; and
drawing the inserted biodegradable multifilament draw-textured yarn to impart bulkiness.

10. The method according to claim 9, wherein the monofilament or multifilament yarn in the preparation of the biodegradable multifilament draw-textured yarn has a single-yarn diameter of 5-30 μm.

11. The method according to claim 9, wherein the multifilament yarn after plying in the preparation of the biodegradable multifilament draw-textured yarn has a diameter of 80-8000 μm.

12. The method according to claim 9, wherein the inserted biodegradable multifilament draw-textured yarn is drawn at a ratio of 5-20%.

13. The method according to claim 9, comprising the step of imparting pores having a size of 10-150 μm to the biodegradable multifilament draw-textured yarn by the drawing step.

14. The method according to claim 9, comprising the step of partially imparting a bulkiness of 150-1000% to the biodegradable multifilament draw-textured yarn by the drawing step.
